(19) United States
(12) Patent Application Publication
(10) Pub. No.: US 2021/0196915 A1
(43) Pub. Date: Jul. 1, 2021

(54) ARTIFICIAL INTELLIGENCE-BASED AUTOMATIC INTUBATION DEVICE AND METHOD OF OPERATING THE SAME

(71) Applicant: INDUSTRY-UNIVERSITY COOPERATION FOUNDATION HANYANG UNIVERSITY, Seoul (KR)

(72) Inventors: Tae Ho LIM, Seoul (KR); Yeongtak SONG, Seoul (KR); Yoonje LEE, Seoul (KR); Hyunggoo KANG, Seoul (KR); Jaehoon OH, Seongnam-si (KR); Youngil CHO, Seoul (KR); Jongbong CHOI, Seoul (KR); Jong Su KIM, Seongnam-si (KR)

(73) Assignee: INDUSTRY-UNIVERSITY COOPERATION FOUNDATION HANYANG UNIVERSITY, Seoul (KR)

(21) Appl. No.: 16/755,726

(22) PCT Filed: Oct. 12, 2018
(86) PCT No.: PCT/KR2018/012066

§ 371 Date: Apr. 13, 2020

(30) Foreign Application Priority Data
Oct. 13, 2017 (KR) 10-2017-0133518
Jul. 25, 2018 (KR) 10-2018-0086804

(51) Int. Cl. A61M 16/04; G06T 7/70; A61B 1/267; A61M 16/00; G05B 13/02; G16H 20/40

(52) U.S. Cl. CPC A61M 16/0418; G06T 7/70; A61B 1/267; A61M 16/022; G05B 13/0265; G06T 2207/20084; G06T 2207/10068; G06T 2207/20081; A61M 2205/505; A61M 2205/50; A61M 2205/3306; G16H 20/40

(57) ABSTRACT

An artificial intelligence-based automatic intubation device according to an exemplary embodiment of the present invention includes: a body having, at one side, a video laryngoscope having an image capturing channel having a predetermined length, the body including a drive unit configured to move an intubation tube; a blade coupled to a circumference of the image capturing channel in a longitudinal direction of the image capturing channel, the blade being configured to be inserted into a trachea; and a control unit configured to control operations of the body and the blade so that the intubation tube is automatically introduced into the trachea.

### TECHNICAL FIELD

The present invention relates to an automatic intubation device and a method of operating the same, and more particularly, to an artificial intelligence-based automatic intubation device and a method of operating the same, which automatically introduce a tube along a respiratory tract detected by means of an artificial intelligence algorithm.

### BACKGROUND ART

Endotracheal intubation is a main technique for saving lives of patients who are in a situation in which spontaneous breathing is impossible or difficult due to difficulty in breathing or a loss of consciousness. Until practitioners master this technique, it is difficult to learn this technique to the extent that it is recommended that the training of this technique needs to be performed in the presence of highly skilled professionals. In addition, it takes a lot of time and effort to master this technique.

Initially, it was known that it is difficult to insert the laryngoscope directly into the trachea. As a kind of laryngoscope to overcome this difficulty, a video laryngoscope has been developed.

The video laryngoscopes may be roughly classified into a channel-type video laryngoscope having a guide channel that guides the laryngoscope into a trachea, a stylet-type video laryngoscope configured to allow a practitioner to adjust a direction in which a procedure is performed in a trachea by using a guide stylet, and a video stylet-type video laryngoscope which is not commonly used but has a video camera attached to a stylet such that no laryngoscope is required.

However, the endotracheal intubation is still one of the most difficult techniques for all medical practitioners in emergency situations.

In the related art, the endotracheal intubation is performed while visually observing the appearance of the larynx through a video screen, but errors frequently occur in this process. In addition, because the anatomical structure related to the larynx is different for each individual, in the case of a patient with a laryngeal structure that is difficult to see with the laryngoscope, it is difficult even for a high-skilled medical practitioner to precisely and accurately change a previous method to a subsequent protocol such as a subsequent method without making a mistake.

As a related art, there is Korean Patent No. 10-1561527 entitled ‘Laryngoscope Embedded with Camera’ (published on Oct. 20, 2015).

### DISCLOSURE

### Technical Problem

Exemplary embodiments of the present invention relate to an artificial intelligence-based automatic intubation device and a method of operating the same, in which a respiratory tract position is detected by applying, to an image of a respiratory tract in a trachea, an artificial intelligence algorithm prepared by performing machine learning on a plurality of respiratory tract images, and intubation is performed by automatically adjusting a traveling direction of a tube based on the detected respiratory tract position.

Technical problems to be solved by the present invention are not limited to the above-mentioned technical problem(s), and other technical problem(s), which are not mentioned above, may be clearly understood by those skilled in the art from the following descriptions.

### Technical Solution

An artificial intelligence-based automatic intubation device according to an exemplary embodiment of the present invention includes: a body having, at one side, a video laryngoscope having an image capturing channel having a predetermined length, the body including a drive unit configured to move an intubation tube; a blade coupled to a circumference of the image capturing channel in a longitudinal direction of the image capturing channel, the blade being configured to be inserted into a trachea; and a control unit configured to control operations of the body and the blade so that the intubation tube is automatically introduced into the trachea.

In addition, an image capturing unit and a light emitting unit may be provided at an end of the image capturing channel according to the exemplary embodiment of the present invention, the image capturing unit may be configured to capture an image of the inside of the trachea, and the light emitting unit may be configured to output a signal in the form of light into the trachea.

In addition, the body according to the exemplary embodiment of the present invention may include a mount unit partially opened so that the video laryngoscope is attachable and detachable.

In addition, the drive unit according to the exemplary embodiment of the present invention may have a space into which the intubation tube is inserted, and the drive unit may include: an opening-closing member configured to be opened or closed so as to fix the intubation tube inserted into the body or separate the intubation tube from the body; and rollers configured to move the intubation tube in an up-down direction.

In addition, a lower portion of the blade according to the exemplary embodiment of the present invention may have a bent part having a predetermined size and bent.

In addition, the blade according to the exemplary embodiment of the present invention may further include a cover member formed along one outer surface of the bent part and having a plate shape having a larger width than the blade.

In addition, the artificial intelligence-based automatic intubation device according to the exemplary embodiment of the present invention may further include a guide unit formed along the other outer surface of the bent part and configured to guide the intubation tube through a space formed as the guide unit is spaced apart from the cover member.

In addition, the blade and the guide unit according to the exemplary embodiment of the present invention may include a plurality of segmental parts connected to at least one adjustment member and configured to be changed independently.

In addition, the body according to the exemplary embodiment of the present invention may include: a first button configured to adjust a movement speed of the intubation tube; and a second button configured to adjust an insertion direction of the intubation tube.

In addition, the intubation tube according to the exemplary embodiment of the present invention may be semi-automatically introduced into the trachea by manipulating the first button and the second button.

In addition, the control unit according to the exemplary embodiment of the present invention may detect a respiratory tract position in respect to an image of the inside of the trachea based on a prepared artificial intelligence algorithm, and the control unit may automatically control a traveling direction of the intubation tube in accordance with the respiratory tract position.

In addition, the control unit according to the exemplary embodiment of the present invention may include: an image collecting unit configured to collect image information in respect to an image of the inside of the trachea which is captured through the image capturing channel; a respiratory tract position detecting unit configured to detect a respiratory tract position by applying, to the collected image information, the artificial intelligence algorithm prepared by performing machine learning on a plurality of respiratory tract images; an image creating unit configured to indicate the detected respiratory tract position in the captured image of the inside of the trachea; and an intubation adjusting unit configured to control, based on the detected respiratory tract position, the operations of the body and the blade so that the traveling direction of the intubation tube is automatically adjusted.

In addition, a method of operating an artificial intelligence-based automatic intubation device according to an exemplary embodiment of the present invention includes: capturing an image of the inside of a trachea by using an image capturing channel provided on a video laryngoscope and collecting image information in respect to the captured image of the inside of the trachea; detecting, by a control unit of the intubation device, a respiratory tract position by applying, to the collected image information, an artificial intelligence algorithm prepared by performing machine learning on a plurality of respiratory tract images; indicating, by the control unit, the detected respiratory tract position on the captured image of the inside of the trachea; and controlling, by the control unit, based on the detected respiratory tract position, an operation of a body of the intubation device and an operation of a blade coupled to the image capturing channel so that a traveling direction of an intubation tube is automatically adjusted.

In addition, the method of operating the artificial intelligence-based automatic intubation device according to the exemplary embodiment of the present invention may further include: semi-automatically adjusting the traveling direction of the intubation tube by operating a button provided on the body of the intubation device in accordance with the respiratory tract position indicated on the captured image of the inside of the trachea.

Other detailed matters of the exemplary embodiment are included in the detailed description and the accompanying drawings.

### Advantageous Effects

According to the exemplary embodiments of the present invention, the respiratory tract position may be detected by applying, to an image of the respiratory tract in the trachea, the artificial intelligence algorithm prepared by performing machine learning on a plurality of respiratory tract images, and the intubation may be performed by automatically adjusting the traveling direction of the tube based on the detected respiratory tract position.

According to the exemplary embodiments of the present invention, it is possible to ensure a patient's safety without performing a separate process of training inexperienced medical practitioners.

### DESCRIPTION OF DRAWINGS

FIG. 1 is a perspective view for schematically explaining an artificial intelligence-based automatic intubation device according to an exemplary embodiment of the present invention.

FIG. 2 is a front view of FIG. 1.

FIG. 3 is a top plan view of FIG. 1.

FIGS. 4A and 4B are perspective views for explaining an operation of a drive unit in a main body according to the exemplary embodiment of the present invention.

FIG. 5A is a perspective view for explaining a blade according to the exemplary embodiment of the present invention.

FIG. 5B is a perspective view for explaining a state in which a blade and an intubation tube according to the exemplary embodiment of the present invention are coupled.

FIG. 6A is a top plan view for explaining a state in which the blade and a video laryngoscope according to the exemplary embodiment of the present invention are coupled.

FIG. 6B is a side view illustrating a state in which the blade, the video laryngoscope, and the intubation tube according to the exemplary embodiment of the present invention are coupled.

FIG. 7 is a block diagram for explaining a structure of a control unit according to the exemplary embodiment of the present invention.

FIG. 8 is a view illustrating a shape of an artificial neural network of a multilayer structure used for machine learning according to the exemplary embodiment of the present invention.

FIG. 9A is a source image illustrating the inside of a trachea according to the exemplary embodiment of the present invention.

FIG. 9B is a respiratory tract image in which only a respiratory tract position is shown in the source image according to the exemplary embodiment of the present invention.

FIG. 9C is an image which additionally shows a respiratory tract position detected by applying, to an image of the inside of a trachea, an artificial intelligence algorithm prepared by performing machine learning on a plurality of respiratory tract images.

### DESCRIPTION OF MAIN REFERENCE NUMERALS OF DRAWINGS

1: Intubation device

10: Video laryngoscope

12: Image capturing channel

20: Intubation tube

100: Body

110: Drive unit

112: Opening-closing member

114: Roller

120: Mount unit

122: Fastening unit

130: Fixing unit

131: Battery unit

132: Power button

134: First button

136: Second button

138: On-off switch

200: Blade

210: Bent part

212: Cover member

220: Guide unit

230: Segmental part

300: Control unit

310: Image collecting unit

320: Respiratory tract position detecting unit

330: Image creating unit

340: Intubation adjusting unit

### BEST MODE

Advantages and/or features of the present invention and methods of achieving the advantages and features will be clear with reference to exemplary embodiments described in detail below together with the accompanying drawings. However, the present invention is not limited to the exemplary embodiments disclosed herein but will be implemented in various forms. The exemplary embodiments of the present invention are provided so that the present invention is completely disclosed, and a person with ordinary skill in the art can fully understand the scope of the present invention. The present invention will be defined only by the scope of the appended claims. Like reference numerals indicate like constituent elements throughout the specification.

Hereinafter, exemplary embodiments of the present invention will be described in detail with reference to the accompanying drawings.

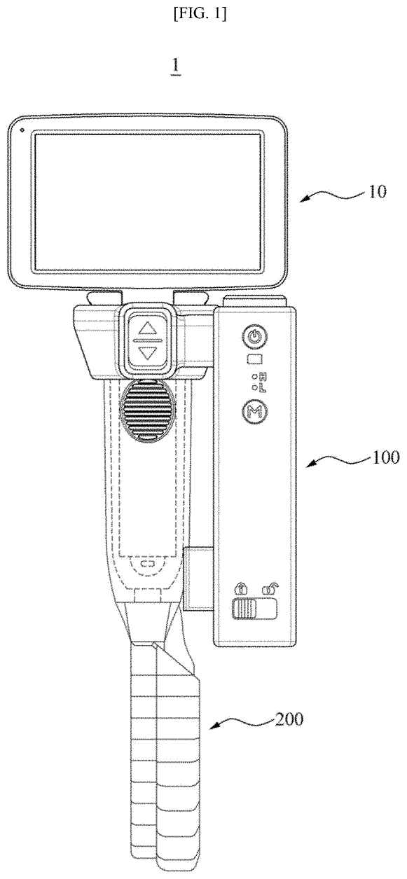

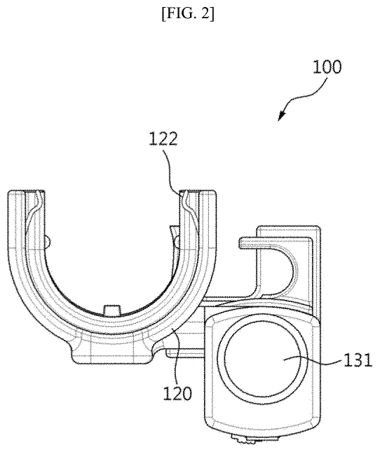

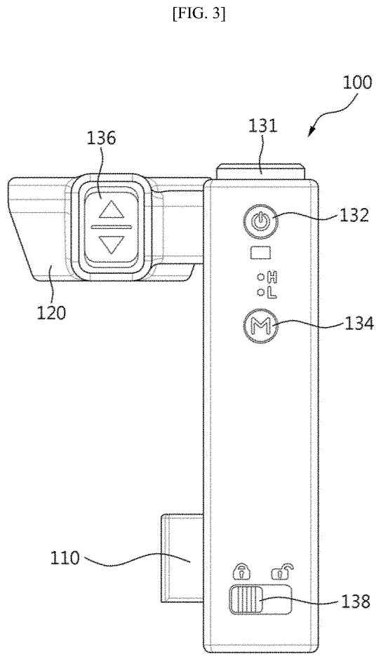

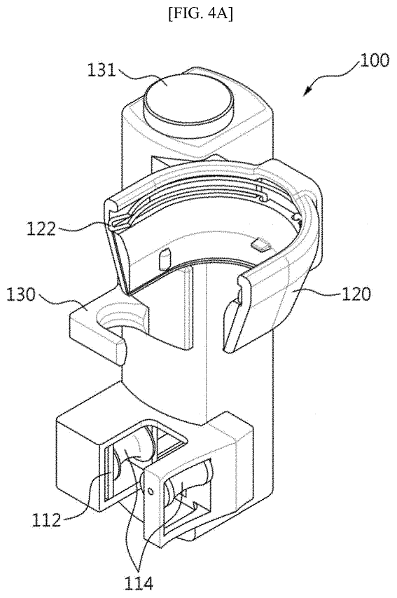

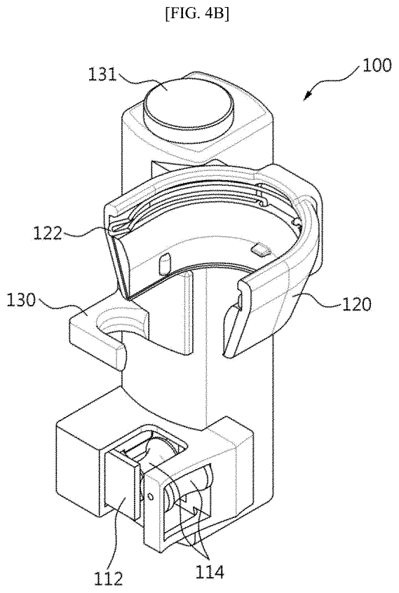

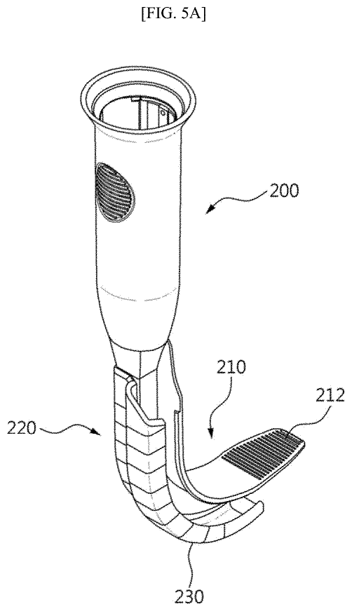

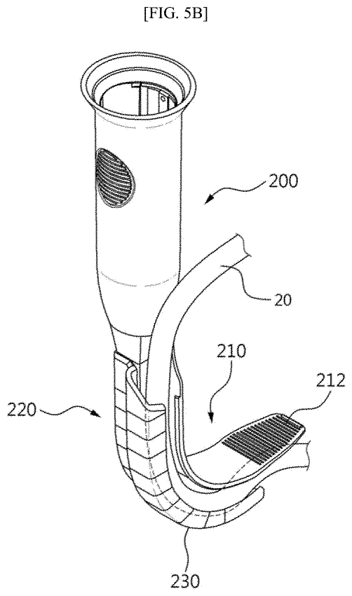

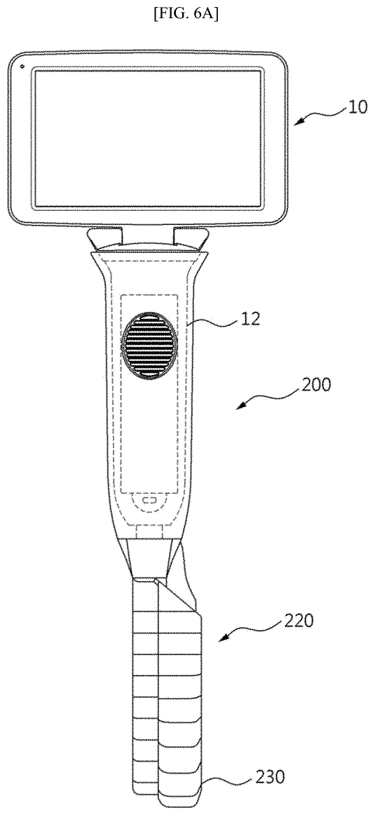

FIG. 1 is a perspective view for schematically explaining an artificial intelligence-based automatic intubation device according to an exemplary embodiment of the present invention, FIG. 2 is a front view of FIG. 1, FIG. 3 is a top plan view of FIG. 1, FIGS. 4A and 4B are perspective views for explaining an operation of a drive unit in a main body according to the exemplary embodiment of the present invention, FIG. 5A is a perspective view for explaining a blade according to the exemplary embodiment of the present invention, FIG. 5B is a perspective view for explaining a state in which a blade and an intubation tube according to the exemplary embodiment of the present invention are coupled, FIG. 6A is a top plan view for explaining a state in which the blade and a video laryngoscope according to the exemplary embodiment of the present invention are coupled, and FIG. 6B is a side view illustrating a state in which the blade, the video laryngoscope, and the intubation tube according to the exemplary embodiment of the present invention are coupled.

Referring to FIG. 1, an artificial intelligence-based automatic intubation device 1 according to an exemplary embodiment of the present invention is a device that performs the intubation by automatically adjusting a traveling direction in which a tube is introduced into a respiratory tract of a patient. The artificial intelligence-based automatic intubation device 1 may include a body 100, a blade 200, and a control unit 300.

As illustrated, the intubation device 1 according to the present invention has a structure in which the blade 200 is coupled to an image capturing channel of a video laryngoscope 10 provided on the body 100, and the automatic intubation is performed through the blade 200.

The video laryngoscope 10 and the intubation tube 20 may be coupled to the body 100, the video laryngoscope 10 is a tool for capturing an image of the inside of a patient's respiratory tract, and the intubation tube 20 is inserted into the respiratory tract.

The video laryngoscope 10 may include a display device configured to display a captured image of the inside of a trachea and an image capturing channel 12 extending from one side of the display device and configured to be inserted into the trachea.

In this case, in a state in which the video laryngoscope 10 is inserted into the blade 200 to be described below, the video laryngoscope 10 may be rotated in all directions, which may improve the convenience of use.

The display device may be implemented as various types screen inquiry devices such as a CRT display, an LCD display, and a PDP display.

In this case, the video laryngoscope 10 may be operated in conjunction with external devices (not illustrated) such as PCs, laptop computers, image projectors, TVs, and smartphones connected to the video laryngoscope 10 by wired or wireless communication. Therefore, the captured image of the inside of the trachea, which is displayed on the display device of the video laryngoscope 10, may be transferred to the external device and displayed through the external device, or data required for the intubation procedure may be received from the external device and displayed through the video laryngoscope 10.

An image capturing unit (not illustrated) and a light emitting unit (not illustrated) may be provided at an end of the image capturing channel 12. That is, the image capturing channel 12 may capture an image of the inside of the trachea by using the image capturing unit and output a signal in the form of light into the trachea by using the light emitting unit so as to enable the image capturing process.

For example, the image capturing unit and the light emitting unit may be disposed in parallel with each other, or a plurality of light emitting units may be disposed along an outer periphery of the image capturing unit. However, the present invention is not limited to a shape in which the image capturing unit and the light emitting unit are disposed.

As the image capturing channel 12 is inserted into the trachea, the intubation tube 20 may be inserted into the respiratory tract in a state in which an airway of the patient's respiratory tract is ensured.

In this case, the intubation tube 20 may be automatically inserted to a respiratory tract position by being controlled by the control unit 300 based on an artificial intelligence algorithm to be described below.

The intubation tube 20 is made of a flexible material and may be curved so as to be inserted while corresponding to a curved shape inside the trachea. However, the present invention is not limited thereto, and the intubation tube 20 may be implemented with various materials such as PVC, silicone, or synthetic resin having elasticity.

Hereinafter, a structure of the body 100 according to the present exemplary embodiment will be specifically described with reference to FIGS. 2, 3, 4A, and 4B.

Referring to FIGS. 2 and 3, the body 100 may include a gripping part (not illustrated) configured to be gripped by a user, a mount unit 120 to/from which the video laryngoscope 10 is attached or detached, and a drive unit 110 configured to move the intubation tube 20.

The gripping part has a column shape having a predetermined length and may be provided with various types of buttons, switches, and indicators which are used to perform the intubation procedure.

The various types of buttons may include a power button 132 configured to turn on or off the body 100, a first button 134 configured to adjust a movement speed of the intubation tube 20, and a second button 136 configured to adjust an insertion direction of the intubation tube 20. However, the present invention is not limited thereto, and any button required for the intubation procedure may be additionally provided.

The first button 134 is implemented as a single button, and the movement speed of the intubation tube 20 may be adjusted in accordance with the number of times the first button 134 is pushed. That is, the movement speed of the intubation tube 20 may be decreased when the first button 134 is pushed once, and the movement speed of the intubation tube 20 may be increased as the first button 134 is continuously pushed twice. However, the present invention is not limited to the number of times the first button 134 is pushed.

In this case, indicators may be further provided which divide movement speeds of the intubation tube 20 into modes in order to visually display the movement speeds of the intubation tube 20. For example, an ‘L’ indicator may emit light for indicating a mode in which the movement speed of the intubation tube 20 is low, and an ‘H’ indicator may emit light for indicating a mode in which the movement speed of the intubation tube 20 is high.

The second button 136 may include a forward button ( [symbol] ) and a reverse button ( [symbol] ) in view of the traveling direction of the intubation tube 20. For example, when the forward button ( [symbol] ) of the second button 136 is pushed, the intubation tube 20 may be moved forward toward the respiratory tract and inserted into the respiratory tract. When the reverse button ( [symbol] ) of the second button 136 is pushed, the intubation tube 20 may be moved reversely to the outside of the respiratory tract and withdrawn from the respiratory tract.

In this case, the first button 134 and the second button 136 may allow a user to manually manipulate the intubation tube 20 to perform the intubation in a case in which it is difficult to automatically insert the intubation tube 20 to perform the automatic intubation function. For example, the user may manually manipulate the buttons when the automatic intubation function is interrupted during the intubation procedure or when an abnormality occurs in the display device of the video laryngoscope 10.

For reference, in the present exemplary embodiment, the automatic intubation procedure using the intubation tube 20 may be started by pushing the power button 132 or a separate drive button (not illustrated).

Meanwhile, the gripping part may be further provided with an on-off switch 138 configured to control an opening-closing operation of the drive unit 110, and a battery unit 131 into which a battery for operating the body 100 is inserted.

The mount unit 120 protrudes from one end of the gripping part, and a part of the mount unit 120 may be opened so that the video laryngoscope 10 may be attached or detached.

In this case, a fastening unit 122 having a clip shape may be provided inside the mount unit 120, and the video laryngoscope 10 may be fastened to the mount unit 120 by means of the fastening unit 122.

For reference, in the present exemplary embodiment, the video laryngoscope 10 may be integrally coupled to the body 100 instead of being attachable to or detachable from the mount unit 120.

The drive unit 110 serves to move the intubation tube 20 toward the inside and the outside of the respiratory tract in the state in which the intubation tube 20 is coupled to the body 100. The drive unit 110 may include an opening-closing member 112 and rollers 114.

The drive unit 110 may have a space having a predetermined size so that the intubation tube 20 is inserted into the space, and the size of the space may be larger than a circumference of the intubation tube 20.

The opening-closing member 112 may be opened or closed so that the intubation tube 20 inserted into the space is fixed to the body 100 or separated from the body 100.

That is, when the opening-closing member 112 is opened as illustrated in FIG. 4A, the intubation tube 20 may be inserted into the space so as to be coupled to the body 100, or the intubation tube 20 may be withdrawn from the space so as to be separated from the body 100. In contrast, when the opening-closing member 112 is closed as illustrated in FIG. 4B, the intubation tube 20 may be fixed to the body 100.

In this case, the opening-closing member 112 may be operated by a drive motor (not illustrated) embedded in the drive unit 110 or disposed outside the drive unit 110, and the drive motor may be operated by the operation of the on-off switch 138 provided in the body 100.

In a state in which one end and the other end of each of the rollers 114 are fixed, the rollers 114 may be rotated so that the intubation tube 20 is moved in an up-down direction. Like the opening-closing member 112, the rollers 114 may be operated by the drive motor (not illustrated).

Meanwhile, the body 100 may be further provided with a fixing unit 130 that fixes the intubation tube 20 so that the intubation tube 20 is moved without swaying.

Referring back to FIG. 1, the blade 200 may be coupled in a longitudinal direction of the image capturing channel so as to surround an outer circumference of the image capturing channel 12.

In the present exemplary embodiment, the blade 200 may be made of a transparent or semi-transparent plastic material so that the image capturing channel 12 coupled inside the blade 200 may be observed with the naked eye. However, the present invention is not limited thereto, and the blade 200 may be made of various materials that enable the inside of the blade 200 to be observed from the outside.

Referring to FIGS. 5A and 5B, a lower portion of the blade 200 may have a bent part 210 which has a predetermined size and is bent. Therefore, irritation to an inner wall of the larynx, which is caused when inserting the blade 200 into the trachea, may be minimized, thereby minimizing discomfort felt by the patient.

The blade 200 may further include a cover member 212 formed along one outer surface of the bent part 210 and having a plate shape having a larger width than the blade 200.

The cover member 212 may have a bent shape formed in a longitudinal direction of the bent part 210 and have the same degree of bending as the bent part 210.

In the present invention, a guide unit 220 may be further formed along the other outer surface of the bent part 210.

The guide unit 220 may be formed in the longitudinal direction of the bent part 210 so as to face the cover member 212.

The guide unit 220 may guide the intubation tube 20 through a space provided as the guide unit 220 is spaced apart from the cover member 212. That is, the space having a predetermined size may be provided between the guide unit 220 and the cover member 212, and the intubation tube 20 may be inserted into the space.

For reference, the guide unit 220 may be integrally formed along the other outer surface of the bent part 210, but in the present invention, the guide unit 220 may also be formed to be separable from the other outer surface of the bent part 210.

Meanwhile, the blade 200 and the guide unit 220 may include a plurality of segmental parts 230, each of which may be connected to at least one adjustment member and changed independently.

The plurality of segmental parts 230 may have a structure similar to a joint structure of a human body and include a plurality of segmental members, such that a traveling direction in which the intubation tube 20 is inserted into the trachea may be freely adjusted.

In this case, the plurality of segmental parts 230 may be connected to at least one adjustment member (not illustrated) in order to change the respective segmental members. The adjustment member may individually adjust angles, curvatures, volumes, and the like of the segmental members, thereby changing the respective segmental members.

For reference, although not illustrated, the adjustment member may be operated by being connected to a motor or pneumatic device and may change the plurality of segmental parts 230 based on the artificial intelligence algorithm to be described below.

Referring to FIGS. 6A and 6B, the image capturing channel 12 may be fitted into the blade 200. Therefore, in the state in which the blade 200 is coupled to the image capturing channel 12, the blade 200 may be inserted into the trachea.

That is, the blade 200 is inserted into the trachea in the state in which the image capturing channel 12 of the video laryngoscope 10 is mounted in the blade 200, and then the intubation tube 20 may be inserted through the guide unit 220. For reference, although not illustrated in the drawings, it can be seen that the image capturing channel 12 penetrates the blade 200 and passes through the end of the bent part 210 of the blade 200.

Referring back to FIG. 1, although not illustrated in the drawings, the control unit 300 may control the operation of the body 100 so that the intubation tube 20 is automatically inserted into the trachea.

The control unit 300 may be a component or a circuit having its own calculation function, and the control unit 300 may be embedded in the body 100 or included in an external device (not illustrated) connected to the body 100 by wired or wireless communication.

The control unit 300 detects a respiratory tract position by applying, to an image of the inside of the trachea, the artificial intelligence algorithm prepared by performing machine learning on a plurality of respiratory tract images, and the control unit 300 may automatically control the traveling direction of the intubation tube 20 based on the respiratory tract position.

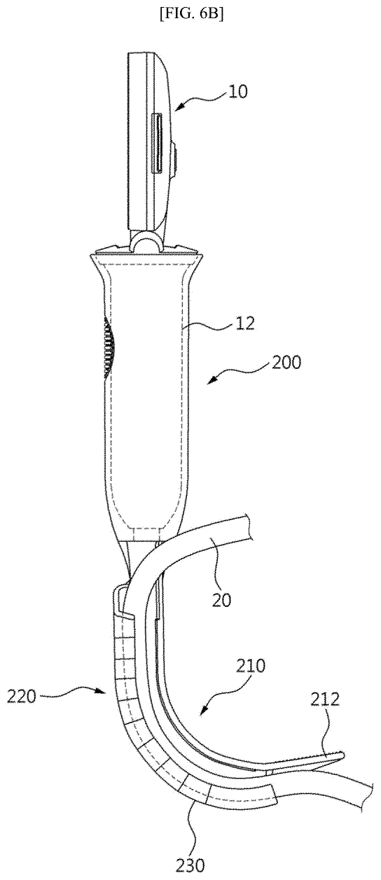

The control unit 300 may not only automatically control the traveling direction of the intubation tube 20, but also may allow the traveling direction of the intubation tube 20 to be controlled semi-automatically or manually. This configuration will be described in detail with reference to FIG. 7.

FIG. 7 is a block diagram for explaining a structure of the control unit according to the exemplary embodiment of the present invention.

Referring to FIG. 7, the control unit 300 may include an image collecting unit 310, a respiratory tract position detecting unit 320, an image creating unit 330, and an intubation adjusting unit 340.

The image collecting unit 310 may collect image information related to the images of the inside of the trachea which are captured through the image capturing channel 12.

That is, the image collecting unit 310 may capture the images of the inside of the trachea several times by using the image capturing unit and the light emitting unit provided in the image capturing channel 12 of the video laryngoscope 10, and the image collecting unit 310 may collect the image information including the plurality of captured images of the inside of the trachea. In this case, the image information is an image file and may include a still image or a video. For example, by using the image capturing channel 12, a video of the inside of the trachea may be captured, a still image is extracted from a file of the captured video, and the still image may be used as an image file.

The image collecting unit 310 may additionally indicate the respiratory tract position detected by applying, to the captured source image, the artificial intelligence algorithm prepared by performing the machine learning on the plurality of respiratory tract images.

In this case, the machine learning for creating the artificial intelligence algorithm may be performed on the external device (not illustrated) connected by wired or wireless communication, and the created artificial intelligence algorithm may be used by being mounted on a chip that may be inserted into the automatic intubation device according to the present invention. That is, according to the present invention, the machine learning is performed by the external device connected to the automatic intubation device, the artificial intelligence algorithm may be prepared in advance, and then the prepared algorithm may be applied to the respiratory tract image.

The respiratory tract position detecting unit 320 may detect the respiratory tract position by applying the artificial intelligence algorithm to the collected image information.

The machine learning is the repetitive learning using an artificial neural network and randomly changes a plurality of internal variables that constitutes the artificial neural network. The machine learning may be repetitively performed in consideration of a relationship between values of the randomly changed internal variables and input and output values of the artificial neural network.

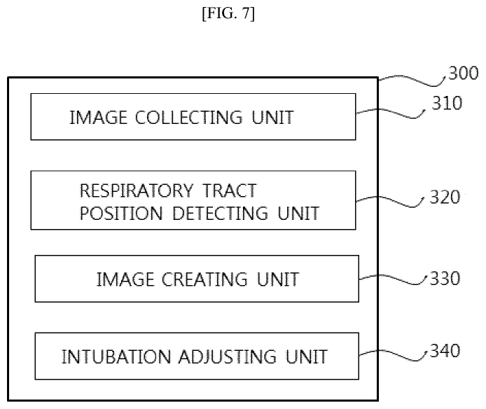

In the present exemplary embodiment, as illustrated in FIG. 8, a multi-layered neural network is designed, and the machine learning is performed by a separate computer based on the plurality of collected respiratory tract images and the respective respiratory tract positions. For example, the multi-layered neural network may be designed by using various types of hidden layers between an input layer and an output layer of the artificial neural network.

The image creating unit 330 may additionally indicate the respiratory tract position detected based on the captured image of the inside of the trachea.

That is, the image creating unit 330 may create an image in which the respiratory tract position, which is detected by applying the artificial intelligence algorithm to the captured source image, is additionally indicated. In this case, the image in which the respiratory tract position, which is detected by applying the artificial intelligence algorithm, is additionally indicated may be displayed on the display device of the video laryngoscope 10 or displayed in real time while performing the respiratory tract intubation.

Based on the respiratory tract position additionally indicated on the image, the intubation adjusting unit 340 may control the operations of the body 100 and the blade 200 so that the traveling direction of the intubation tube 20 may be automatically adjusted.

Therefore, the intubation tube 20 may be automatically introduced to the corresponding respiratory tract position by applying the respiratory tract position indicated on the image by the machine learning.

Meanwhile, the intubation tube 20 according to the present invention may be introduced in accordance with three types of exemplary embodiments.

In one exemplary embodiment, as an automatic mode, the blade 200, particularly, the plurality of segmental parts 230 is automatically moved by the above-mentioned process based on the artificial intelligence algorithm, such that the traveling direction of the intubation tube 20 may be automatically controlled in accordance with the respiratory tract position, and thus the movement of the intubation tube 20 itself, such as the insertion of the intubation tube 20 into the trachea or the withdrawal of the intubation tube 20 from the trachea, may be automatically controlled by the control unit 300.

In another exemplary embodiment, as a semi-automatic mode, the blade 200, particularly, the plurality of segmental parts 230 is automatically moved by the above-mentioned process based on the artificial intelligence algorithm, such that the traveling direction of the intubation tube 20 is automatically controlled in accordance with the respiratory tract position, and thus the movement of the intubation tube 20, such as the insertion of the intubation tube 20 into the trachea or the withdrawal of the intubation tube 20 from the trachea, may be manually controlled by using the various types of buttons provided on the body 100 of the intubation device.

In still another exemplary embodiment, as a semi-automatic mode, the blade 200, particularly, the plurality of segmental parts 230 is automatically moved by the above-mentioned process based on the artificial intelligence algorithm, such that the traveling direction of the intubation tube 20 may be automatically controlled in accordance with the respiratory tract position, and thus the movement of the intubation tube 20, such as the insertion of the intubation tube 20 into the trachea or the withdrawal of the intubation tube 20 from the trachea, may be manually controlled by the user by directly manipulating the intubation tube 20.

In yet another exemplary embodiment, as a manual mode, the traveling direction of the intubation tube 20 is manually controlled by the user by directly manipulating the intubation tube 20, such that the movement of the intubation tube 20, such as the insertion of the intubation tube 20 into the trachea or the withdrawal of the intubation tube 20 from the trachea, may also be manually controlled by the user by directly manipulating the intubation tube 20.

Hereinafter, a method of operating the artificial intelligence-based automatic intubation device according to the exemplary embodiment of the present invention will be described.

First, the automatic intubation device 1 according to the present invention may capture the image of the inside of the trachea by using the image capturing channel 12 provided on the video laryngoscope 10 which is attachable to or detachable from the intubation device 1. The image of the inside of the trachea is an image that represents the inside of the respiratory tract, that is, an image used for the artificial intelligence algorithm for detecting the respiratory tract position.

In this case, the automatic intubation device 1 may capture images of the inside of the trachea several times by using the image capturing unit and the light emitting unit provided on the image capturing channel 12 and may collect the image information related to the plurality of captured images.

In addition, the automatic intubation device 1 may create a respiratory tract image in which the respiratory tract position detected by the artificial intelligence algorithm is additionally indicated in the captured source image.

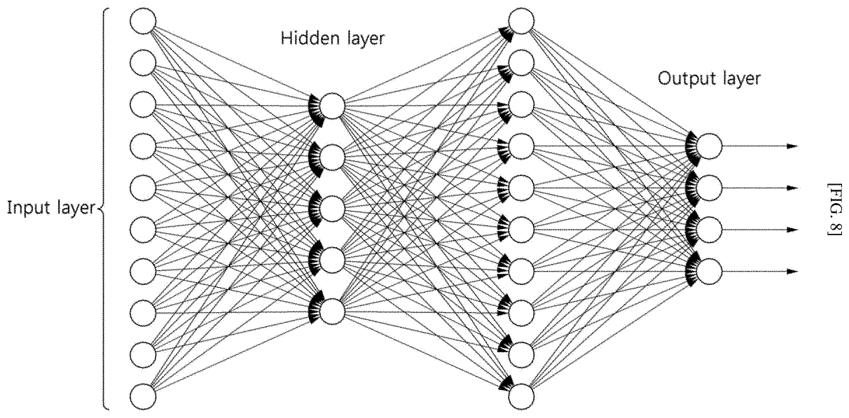

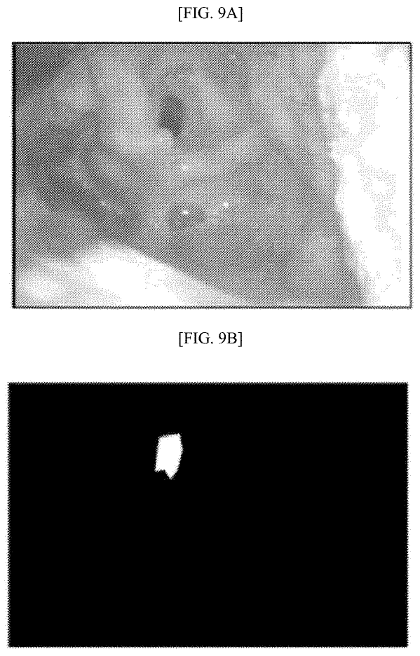

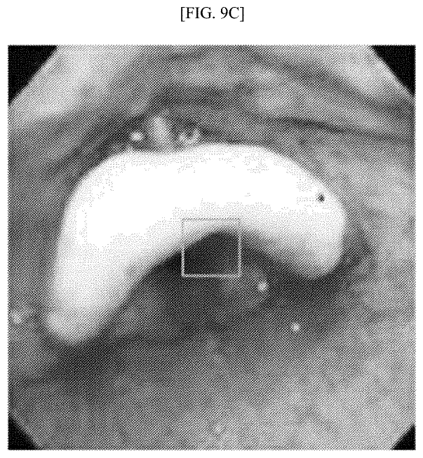

For reference, an example of the source image, which indicates the inside of the trachea, is as illustrated in FIG. 9A, and an example of the respiratory tract image (white), which indicates only the respiratory tract position on the source image, is as illustrated in FIG. 9B.

Therefore, the artificial intelligence algorithm for detecting the respiratory tract position on the respiratory tract image may be prepared by performing the machine learning on the plurality of respiratory tract images.

Next, the automatic intubation device I may detect the respiratory tract position by applying the prepared artificial intelligence algorithm to the respiratory tract image collected by the control unit 300.

Specifically, pixel values of the source images and the respiratory tract images in respect to the collected image information may be converted into numerical value data having values between 0 and 1 by using a black-white conversion formula ((Red×0.299+Green×0.587+Blue×0.114)/255).

In this case, the numerical value data converted from the source image may be used as input values for the machine learning, and the numerical value data converted from the respiratory tract image may be used as target values for the machine learning.

The machine learning according to the present exemplary embodiment is the repetitive learning using the artificial neural network and randomly selects some of the plurality of internal variables that constitutes the artificial neural network, thereby randomly changing the corresponding variable values.

Thereafter, the input values, which are numerical value data converted from the source image, are inputted as the input values for the machine learning, such that the output values of the artificial neural network, which have the randomly changed internal variable values, may be calculated.

Thereafter, based on the calculated output values, the calculation is performed to square and sum differences between the target value and the numerical value data converted from the respiratory tract image.

Thereafter, when the squared and summed values are smaller than before randomly changing the internal variable values of the artificial neural network, the changed internal variable values are stored in the artificial neural network. When the squared and summed values are larger than before randomly changing the internal variable values of the artificial neural network, the changed internal variable values are not stored, and the machine learning is repetitively performed. In this case, a position of a maximum output value of the artificial neural network, which corresponds to the stored internal variable value, may be set as the respiratory tract position indicated in the image.

For reference, the number of times the machine learning is repeated is set in advance, and the machine learning is repetitively performed until the set number of times is reached.

Next, the automatic intubation device 1, by the control unit 300, may create an image in which the respiratory tract position, which is detected by applying the artificial intelligence algorithm, is additionally indicated on the captured image of the inside of the trachea.

In this case, an example of the image in which the respiratory tract position (green quadrangle), which is detected by applying the artificial intelligence algorithm, is additionally indicated on the image of the inside of the trachea is as illustrated in FIG. 9C.

For reference, the image in which the respiratory tract position, which is detected by applying the artificial intelligence algorithm prepared in respect to the respiratory tract image, is additionally indicated may be displayed on the display device of the video laryngoscope 10 or displayed in real time while performing the respiratory tract intubation.

Next, the automatic intubation device I may control the operations of the body 100 and the blade 200 so that the traveling direction of the intubation tube 20 is adjusted based on the respiratory tract position detected by applying the artificial intelligence algorithm by the control unit 300.

Specifically, the intubation tube 20 is moved along the guide unit 220 provided on the blade 200, and the angles, the curvatures, the volumes, and the like of the plurality of segmental parts 230, which constitutes the guide unit 220 and the blade 200, are adjusted, such that the traveling direction of the intubation tube 20 may be adjusted so that the intubation tube 20 is moved toward the respiratory tract position.

Accordingly, the intubation tube 20 may be automatically introduced toward the corresponding respiratory tract position based on the respiratory tract position detected by applying the artificial intelligence algorithm to the respiratory tract image.

Thereafter, after the machine learning is performed a predetermined number of times, the operation of the intubation device is ended.

While the specific exemplary embodiments according to the present invention have been described above, various modifications may be made without departing from the scope of the present invention. Therefore, the scope of the present invention should not be limited to the described exemplary embodiments and should be defined by not only the claims to be described below, but also those equivalent to the claims.

While the present invention has been described above with reference to the limited exemplary embodiments and the drawings, the present invention is not limited to the exemplary embodiments and may be variously modified and altered from the disclosure by those skilled in the art to which the present invention pertains. Therefore, the spirit of the present invention should be defined only by the appended claims, and all modifications, equivalents, and alternatives fall within the scope and spirit of the present invention.

1. An artificial intelligence-based automatic intubation device comprising:
a body having, at one side, a video laryngoscope having an image capturing channel having a predetermined length, the body comprising a drive unit configured to move an intubation tube;
a blade coupled to a circumference of the image capturing channel in a longitudinal direction of the image capturing channel, the blade being configured to be inserted into a trachea; and
a control unit configured to control operations of the body and the blade so that the intubation tube is automatically introduced into the trachea.

2. The artificial intelligence-based automatic intubation device of claim 1, wherein an image capturing unit and a light emitting unit are provided at an end of the image capturing channel, the image capturing unit is configured to capture an image of the inside of the trachea, and the light emitting unit is configured to output a signal in the form of light into the trachea.

3. The artificial intelligence-based automatic intubation device of claim 1, wherein the body comprises a mount unit partially opened so that the video laryngoscope is attachable and detachable.

4. The artificial intelligence-based automatic intubation device of claim 1, wherein the drive unit has a space into which the intubation tube is inserted, and the drive unit comprises:
an opening-closing member configured to be opened or closed so as to fix the intubation tube inserted into the body or separate the intubation tube from the body; and
rollers configured to move the intubation tube in an up-down direction.

5. The artificial intelligence-based automatic intubation device of claim 1, wherein a lower portion of the blade has a bent part having a predetermined size and bent.

6. The artificial intelligence-based automatic intubation device of claim 5, wherein the blade further comprises a cover member formed along one outer surface of the bent part and having a plate shape having a larger width than the blade.

7. The artificial intelligence-based automatic intubation device of claim 6, further comprising:
a guide unit formed along the other outer surface of the bent part and configured to guide the intubation tube through a space formed as the guide unit is spaced apart from the cover member.

8. The artificial intelligence-based automatic intubation device of claim 7, wherein the blade and the guide unit comprise a plurality of segmental parts connected to at least one adjustment member and configured to be changed independently.

9. The artificial intelligence-based automatic intubation device of claim 1, wherein the body comprises:
a first button configured to adjust a movement speed of the intubation tube; and
a second button configured to adjust an insertion direction of the intubation tube.

10. The artificial intelligence-based automatic intubation device of claim 9, wherein the intubation tube is semi-automatically introduced into the trachea by manipulating the first button and the second button.

11. The artificial intelligence-based automatic intubation device of claim 1, wherein the control unit detects a respiratory tract position in respect to an image of the inside of the trachea based on a prepared artificial intelligence algorithm, and the control unit automatically controls a traveling direction of the intubation tube in accordance with the respiratory tract position.

12. The artificial intelligence-based automatic intubation device of claim 11, wherein the control unit comprises:
an image collecting unit configured to collect image information in respect to an image of the inside of the trachea which is captured through the image capturing channel;
a respiratory tract position detecting unit configured to detect a respiratory tract position by applying, to the collected image information, the artificial intelligence algorithm prepared by performing machine learning on a plurality of respiratory tract images;
an image creating unit configured to indicate the detected respiratory tract position in the captured image of the inside of the trachea; and
an intubation adjusting unit configured to control, based on the detected respiratory tract position, the operations of the body and the blade so that the traveling direction of the intubation tube is automatically adjusted.

13. A method of operating an artificial intelligence-based automatic intubation device, the method comprising:
capturing an image of the inside of a trachea by using an image capturing channel provided on a video laryngoscope and collecting image information in respect to the captured image of the inside of the trachea;
detecting, by a control unit of the intubation device, a respiratory tract position by applying, to the collected image information, an artificial intelligence algorithm prepared by performing machine learning on a plurality of respiratory tract images;
indicating, by the control unit, the detected respiratory tract position on the captured image of the inside of the trachea; and
controlling, by the control unit, based on the detected respiratory tract position, an operation of a body of the intubation device and an operation of a blade coupled to the image capturing channel so that a traveling direction of an intubation tube is automatically adjusted.

14. The method of claim 13, further comprising:
semi-automatically adjusting the traveling direction of the intubation tube by operating a button provided on the body of the intubation device in accordance with the respiratory tract position indicated on the captured image of the inside of the trachea.
